(19) United States
(12) Patent Application Publication
(10) Pub. No.: US 2014/0294921 A1
(43) Pub. Date: Oct. 2, 2014

(54) COMPOSITION FOR TIPICAL USE FOR TREATING SKIN DISORDERS

(71) Applicants: Aleardo Koverech, Rome (IT); Ashraf Virmani, Ariccia (Rome) (IT)

(72) Inventors: Aleardo Koverech, Rome (IT); Ashraf Virmani, Ariccia (Rome) (IT)

(73) Assignee: SIGMA-TAU INDUSTRIE FAMACEUTICHE RIUNITE S.P.A., Rome (IT)

(21) Appl. No.: 13/992,294

(22) PCT Filed: Nov. 28, 2011
(86) PCT No.: PCT/EP11/71151

§ 371 Date: Jun. 27, 2013

(30) Foreign Application Priority Data
Dec. 9, 2010 (EP) 10194329.8

(51) Int. Cl. A61K 31/4188; A61K 31/714; A61K 31/045; A61K 31/16; A61K 31/205; A61K 31/355

(52) U.S. Cl. CPC A61K 31/4188; A61K 31/205; A61K 31/355; A61K 31/045; A61K 31/16; A61K 31/714
USPC 424/450; 514/387; 514/52; 424/94.1

(57) ABSTRACT

It is described a composition, topically administrable, comprising as active ingredients L-carnitine or a derivative thereof, biotin, and at least one or more of the following active ingredients: vitamin B 12; vitamin E; bisabolol; and panthenol; useful for treating skin disorders.

The present invention relates to a pharmaceutical or cosmetic composition, topically administrable, useful for preventing and/or treating non-inflammatory skin disorder; inflammatory skin disorder; or degenerative skin disorders such as erythematous, inflammatory, allergic or autoimmune-reactive symptoms, comprising as active ingredients L-carnitine and/or one or more alkanoyl L-carnitines, or mixture thereof, or one of their pharmaceutically acceptable salts; biotin and one ore more of the following active ingredients: vitamin B12; vitamin E; bisabolol; and panthenol.

### FIELD OF THE INVENTION

Inflammatory skin diseases affect men, women, and children of all ages and races. These diseases can be very distressing, causing great discomfort, emotional stress and feelings of hopelessness. Inflammatory skin diseases are often disfiguring. Patients with skin disease often shy away from meaningful social relationships.

Inflammatory skin disease is a broad category that includes many conditions, ranging in severity from mild itching to serious medical health complications.

These conditions include:

Eczema

Also known as Atopic Dermatitis, this disorder causes the skin to become dry, itchy and inflamed. There may be oozing or a crusted appearance.

Acne

An inflammatory skin disease characterized by comedones, papules, pustules and inflamed nodules.

Hives (Also Known as Urticaria)

Hives is an outbreak of red bumps or patches called wheals on the skin. These bumps appear suddenly anywhere on the body and are usually accompanied with an intense itch.

Poison Ivy

An inflammatory allergic rash which develops after contact with the poisonous plant;

Psoriasis

An inflammatory skin disease characterized by dry pink scaling round patches, and

Rosacea

An inflammatory skin disease characterized by flushing and redness appearing in the central area of the face. Pimples and Pustules may appear.

Carnitine is a quaternary ammonium compound biosynthesized from the amino acids lysine and methionine. In living cells, it is required for the transport of fatty acids from the cytosol into the mitochondria during the breakdown of lipids (fats) for the generation of metabolic energy. It is often sold as a nutritional supplement. Carnitine was originally found as a growth factor for mealworms and labeled vitamin Bt. Carnitine exists in two stereoisomers: Its biologically active form is L-carnitine, whereas its enantiomer, D-carnitine, is biologically inactive.

Biotin is a water-soluble B-complex vitamin (vitamin B₇) it is a coenzyme in the metabolism of fatty acids and leucine, and it plays a role in gluconeogenesis.

Biotin is necessary for cell growth, the production of fatty acids, and the metabolism of fats and amino acids. It plays a role in the citric acid cycle, which is the process by which biochemical energy is generated during aerobic respiration. Biotin not only assists in various metabolic reactions but also helps to transfer carbon dioxide. Biotin is often recommended for strengthening hair and nails. Consequently, it is found in many cosmetics and health products for the hair and skin, though it cannot be absorbed through the hair or skin itself.

Biotin deficiency is rare, because intestinal bacteria generally produce biotin in excess of the body's daily requirements. For that reason, statutory agencies in many countries, for example the USA and Australia, do not prescribe a recommended daily intake of biotin.

Vitamin B₁₂ also called cobalamin, is a water soluble vitamin with a key role in the normal functioning of the brain and nervous system, and for the formation of blood. It is one of the eight B vitamins. It is normally involved in the metabolism of every cell of the human body, especially affecting DNA synthesis and regulation, but also fatty acid synthesis and energy production.

Vitamin E is a generic term for tocopherols and tocotrienols. Vitamin E is a fat-soluble antioxidant that stops the production of reactive oxygen species formed when fat undergoes oxidation. Of these alpha-tocopherol has been most studied as it has the highest bioavailability.

It has been claimed that α-tocopherol is the most important lipid-soluble antioxidant, and that it protects cell membranes from oxidation by reacting with lipid radicals produced in the lipid peroxidation chain reaction. This would remove the free radical intermediates and prevent the oxidation reaction from continuing. The oxidised α-tocopheroxyl radicals produced in this process may be recycled back to the active reduced form through reduction by other antioxidants, such as ascorbate, retinol or ubiquinol. However, the importance of the antioxidant properties of this molecule at the concentrations present in the body are not clear and it is possible that the reason why vitamin E is required in the diet is unrelated to its ability to act as an antioxidant.

Bisabolol, or more formally α-(−)-bisabolol or also known as levomenol, is a natural monocyclic sesquiterpene alcohol. It is a colorless, viscous, oil that is the primary constituent of the essential oil from German chamomile (Matricaria recutita) and Myoporum grassifolium. It is almost insoluble in water and glycerin, but well soluble in ethanol. The enantiomer, α-(+)-bisabolol, is also found naturally but is rare. Synthetic bisabolol is usually a racemic mixture of the two, α-(±)-bisabolol.

Bisabolol has a weak sweet floral aroma and is used in various fragrances. It has also been used for hundreds of years in cosmetics because of its perceived skin healing properties. Bisabolol is known to have anti-irritant, anti-inflammatory and anti-microbial properties. Bisabolol is also demonstrated to enhance the percutaneous absorption of certain molecules.

Panthenol is the alcohol analog of pantothenic acid (vitamin B₅), and is thus a provitamin of B₅. In organisms it is quickly oxidized to pantothenate. Panthenol is a highly viscous transparent liquid at room temperature. It is soluble in water, alcohol and propylene glycol, soluble in ether and chloroform, and slightly soluble in glycerin.

Panthenol comes in two enantiomers, D and L. Only D-panthenol (dexpanthenol) is biologically active, however both forms have moisturizing properties. For cosmetic use, panthenol comes either in D form, or as a racemic mixture of D and L (DL-panthenol).

In cosmetics, panthenol is a humectant, emollient and moisturizer. It binds to the hair shaft readily and is a frequent component of shampoos and hair conditioners (in concentrations of 0.1-1%). It coats the hair and seals its surface, lubricating the hair shaft and making strands appear shiny.

In ointments, Panthenol has good skin penetration. It is used for treatment of sunburns, mild burns and minor skin disorders. It improves hydration, reduces itching and inflammation of the skin and accelerates and improves healing of epidermal wounds.

Previous uses of carnitines and biotin in the dermatological field are already known.

WO0209652A2 describes the use of (A) carnitine and/or at least one acylcarnitine, in combination with a compound or a number of compounds from group (B) formed by biotin, lipoic acid, conjugated fatty acids, carnosine, bioquinones, phytofluene, phytoene and folic acid, in cosmetic or dermatological preparations for the treatment and prophylaxis of disturbances of the skin.

Further uses of carnitine are also known.

In Res 1992; 18(8):355-365 the use of L-carnitine in the cardiological field is described.

U.S. Pat. No. 5,543,556 describes the use of acyl L-carnitine esters with gamma-hydroxybutyric acid for the inhibition of neuronal degeneration and in the treatment of coma.

U.S. Pat. No. 5,811,457 describes the use of propionyl L-carnitine for the treatment of chronic obliterating arteriopathy.

The main topical agents used for treating skin disorders are anti-inflammatory agents such as corticosteroids, and anti-infective agents such as antibiotics, antifungals and antiviruses agents.

Topical corticosteroid use, a common and often efficacious therapy for a wide variety of cutaneous conditions, may have substantial adverse effects. These range from the notable nondermatologic side effects of hypothalamic-pituitary-adrenal axis suppression, Cushing's disease, femoral head osteonecrosis, and cataracts, to a variety of less serious skin effects such as cutaneous tinea and contact dermatitis. The broad availability, efficacy, relative low cost, and ease of applying topical corticosteroids should not induce complacency in prescribers. Physicians should have the same awareness of the possible side effects of topical steroid use as when prescribing parenteral medication.

It is well known in the art that also antibiotics, antifungals and antiviruses agents are endowed with adverse effects which limit their use.

The new composition of the invention is composed of active ingredient which do not show the adverse effects of the active ingredients mentioned above.

In the cosmetics and dermatological field it is still a perceived need to have new dermatological compositions useful for treating skin disorders, which are not endowed by the adverse effects of the cosmetic/therapeutical agents used in the art.

### DETAILED DESCRIPTION OF THE INVENTION

It has now been found that L-carnitine and/or one or more alkanoyl L-carnitines, or mixture thereof, or one of their pharmaceutically acceptable salts; biotin and one ore more of the following active ingredients: vitamin B12; vitamin E; bisabolol; and panthenol, are useful agent for preventing and/or treating inflammatory and/or degenerative disturbances of the skin.

It is therefore an object of the present invention a pharmaceutical or cosmetic composition, topically administrable, useful for preventing and/or treating inflammatory and/or degenerative skin disturbances, comprising as active ingredients L-carnitine and/or one or more alkanoyl L-carnitines, or mixture thereof, or one of their pharmaceutically acceptable salts; biotin and one ore more of the following active ingredients: vitamin B12; vitamin E; bisabolol; and panthenol; in which:
L-carnitine or a derivative thereof is present in an amount of from 0.1 to 20% by weight; preferably in an amount of 1 to 10% by weight, most preferably in an amount of 5% by weight;
biotin or a derivative thereof is present in an amount of from 0.01 to 20% by weight; preferably in an amount of 0.1 to 10% by weight, most preferably in an amount of 1% by weight;
vitamin B₁₂ or a derivative thereof is present in an amount of from 0.001 to 10% by weight; preferably in an amount of 00.1 to 1% by weight, most preferably in an amount of 0.1% by weight;
vitamin E or a derivative thereof is present in an amount of from 0.01 to 10% by weight; preferably in an amount of 0.1 to 5% by weight, most preferably in an amount of 0.5% by weight;
bisabolol or a derivative thereof is present in an amount of from 0.01 to 20% by weight; preferably in an amount of 0.1 to 10% by weight, most preferably in an amount of 1% by weight;
panthenol or a derivative thereof is present in an amount of from 0.01 to 20% by weight; preferably in an amount of 0.1 to 10% by weight, most preferably in an amount of 1% by weight.

The composition of the invention may further comprise coenzymes, antioxidants, vitamins, mineral salts and solar filter.

It is a further object of the present invention a pharmaceutical or cosmetic composition, topically administrable, useful for preventing and/or treating inflammatory and/or degenerative skin disturbances, comprising as active ingredients L-carnitine and/or one or more alkanoyl L-carnitines, or mixture thereof, or one of their pharmaceutically acceptable salts; biotin and vitamin B12.

It is a further object of the present invention a pharmaceutical or cosmetic composition, topically administrable, useful for preventing and/or treating inflammatory and/or degenerative skin disturbances, comprising as active ingredients L-carnitine and/or one or more alkanoyl L-carnitines, or mixture thereof, or one of their pharmaceutically acceptable salts; biotin; vitamin E; bisabolol; and panthenol.

It is a further object of the present invention a method for preventing and/or treating inflammatory and/or degenerative skin disturbances, which comprises the topical administration to a patient in need thereof a suitable amount of L-carnitine and/or one or more alkanoyl L-carnitines, or mixture thereof, or one of their pharmaceutically acceptable salts; biotin and one ore more of the following active ingredients: vitamin B12; vitamin E; bisabolol; and panthenol.

The composition of the invention is useful for preventing or treating harmful oxidative processes in the skin, or for prophylaxis of erythematous, inflammatory, allergic or autoimmune-reactive symptoms, in particular dermatitis and dermatoses.

The composition of the invention may be in the form of ointments, creams, lotions baths, solutions powders gels, or in liposome form. Other possible formulations known in the art are also comprised in the scopes of the present invention.

What is meant by pharmaceutically acceptable salt of L-carnitine is any salt with an acid that does not give rise to toxic or side effect.

These acids are well known to pharmacologists and to experts in pharmacy. Non-limiting examples of such salts are: chloride, bromide, orotate, aspartate, acid aspartate, acid citrate, magnesium citrate, phosphate, acid phosphate, fumarate and acid fumarate, magnesium fumarate, lactate, maleate and acid maleate, oxalate, acid oxalate, pamoate, acid pamoate, sulphate, acid sulphate, glucose phosphate, tartrate and acid tartrate, glycerophosphate, mucate, magnesium tartrate, 2-amino-ethanesulphonate, magnesium 2-amino-ethanesulphonate, methanesulphonate, choline tartrate, trichloroacetate, and trifluoroacetate.

What is meant by pharmaceutically acceptable salt of L-carnitine is also a salt approved by the FDA and listed in the publication Int. J. of Pharm. 33 (1986), 201-217, which is incorporated herein by way of a reference.

The following examples further illustrates the invention without limiting it.

### Example 1

### Dithranol-Induced Mouse Ear Inflammation Test

Dithranol is a known inflammatory agent, which induces inflammatory response by releasing free radicals.

The modulatory effects of the compositions of the present invention were tested using the dithranol-induced mouse ear inflammation test described in “Methods in Molecular Biology, 2003, Vol 225; 129-137”, and in “Br. J. Pharmacol. 1991, 104; 990-994”.

Swiss male mice (10 per group) of 6 to 8 weeks old; weighing 28-32 g each; were used. Mice had free access to food and water.

The compounds of the invention were dissolved in water or ethanol (depending on their solubility). 20 μL of the solution containing the compound of the invention, or their combinations, was painted on the left ear (back and front; 10+10 μL) using a Hamilton syringe within 30 seconds (Time 0) The compound tested, and their combinations, are reported in the following

Table A.

TABLE A

| N^{o} | Treatment |
|---|---|
| 1 | Control group (inflamed ear) |
| | (Dithranol, 30 μg in 20 μL of acetone) |
| 2 | L-carnitine (inflamed-treated ear) |
| | (dissolved in water; 0.5 mg in 20 μL) |
| 3 | Biotin (dissolved in water; 0.1 mg in 20 μL) |
| 4 | Vitamin B12 (dissolved in water; 0.01 mg in 20 μL) |
| 5 | Vitamin E (dissolved in ethanol; 0.05 mg in 20 μL) |
| 6 | Bisabolol (dissolved in ethanol; 0.1 mg in 20 μL) |
| 7 | Panthenol (dissolved in water; 0.1 mg in 20 μL) |
| 8 | L-carnitine + biotin (0.5 mg + 0.1 mg in 20 μL) |
| 9 | L-carnitine + vitamin B12 |
| 10 | L-carnitine + vitamin E |
| 11 | L-carnitine + bisabolol |
| 12 | L-carnitine + panthenol |
| 13 | Biotin + vitamin B12 |
| 14 | Biotin + vitamin E |
| 15 | Biotin + bisabolol |
| 16 | Biotin + panthenol |
| 17 | Vitamin B12 + vitamin E |
| 18 | Vitamin B12 + bisabolol |
| 19 | Vitamin B12 + panthenol |
| 20 | Vitamin E + bisabolol |
| 21 | Vitamin E + panthenol |
| 22 | Bisabolol + panthenol |
| 23 | L-carnitine + biotin + vitamin B12 |
| 24 | L-carnitine + biotin + vitamin E |
| 25 | L-carnitine + biotin + vitamin B12 + bisabolol |
| 26 | L-carnitine + biotin + vitamin E + bisabolol |
| 27 | L-carnitine + biotin + vitamin B12 + panthenol |
| 28 | L-carnitine + biotin + vitamin E + panthenol |
| 29 | L-carnitine + biotin + vitamin B12 + Bisabolol + |
| | panthenol |
| 30 | L-carnitine + biotin + vitamin E + bisabolol + |
| | panthenol |

A similar volume of vehicle (water or ethanol) was applied to the other ear (control ear).

After 30 minutes (Time+0.5 h) the irritant, dithranol, 30 μg in 20 μL of acetone was painted on the left ear (back and front, 10+10 μL).

A similar volume of acetone was applied to the other ear.

A group of mice treated only with dithranol (no treatment with the compounds of the invention) was present (control group).

An electric hair dryer for a few seconds to evaporate the solvent was used.

The compounds of the invention were administered again 16 hours from the first treatment (Time+16 h).

After 24 hours from the first treatment (Time+24 h) the animals were killed with an overdose of diethyl ether and the ears were cut off.

A disk from the middle part of each ear was cut off, using a sharp punch 6 mm in diameter.

The disks samples were weighted on a suitable balance, and the extent of the edema (as percentage of inhibition) was calculated according to the formula reported on page 991, right column, of “Br. J. Pharmacol. 1991, 104; 990-994” mentioned above.

The results obtained are reported in Table 1.

TABLE 1
Dithranol-induced mouse ear inflammation test. % of
inhibition.

| | | | % | P< | P< | P< |
|---|---|---|---|---|---|---|
| Group | N^{o} | Treatment | Inhibit. | Vs A | Vs B | Vs C |
| A | 1 | Control | 0 | — | — | — |
| Control | | | | | | |
| B | 2 | L-carnitine | 9 | NS | — | — |
| | 3 | Biotin | 6 | NS | — | — |
| | 4 | Vitamin B12 | 8 | NS | — | — |
| | 5 | Vitamin E | 8 | NS | — | — |
| | 6 | Bisabolol | 6 | NS | — | — |
| | 7 | Panthenol | 7 | NS | — | — |
| C | 8 | L-carnitine + biotin | 17 | NS | — | — |
| | 9 | L-carnitine + vitamin B12 | 18 | NS | — | — |
| | 10 | L-carnitine + vitamin E | 16 | NS | — | — |
| | 11 | L-carnitine + bisabolol | 19 | NS | — | — |
| | 12 | L-carnitine + panthenol | 17 | NS | — | — |
| | 13 | Biotin + vitamin B12 | 18 | NS | — | — |
| | 14 | Biotin + vitamin E | 15 | NS | — | — |
| | 15 | Biotin + bisabolol | 17 | NS | — | — |
| | 16 | Biotin + panthenol | 19 | NS | — | — |
| | 17 | Vitamin B12 + vitamin E | 15 | NS | — | — |
| | 18 | Vitamin B12 + bisabolol | 14 | NS | — | — |
| | 19 | Vitamin B12 + panthenol | 18 | NS | — | — |
| | 20 | Vitamin E + bisabolol | 17 | NS | — | — |
| | 21 | Vitamin E + panthenol | 17 | NS | — | — |
| | 22 | Bisabolol + panthenol | 15 | NS | — | — |
| D | 23 | L-carnitine + biotin + | 83 | 0.001 | 0.001 | 0.001 |
| | | vitamin B12 | | | | |
| | 24 | L-carnitine + biotin + | 60 | 0.001 | 0.001 | 0.01 |
| | | vitamin E | | | | |
| | 25 | L-carnitine + biotin + | 76 | 0.001 | 0.001 | 0.001 |
| | | vitamin B12 + bisabolol | | | | |
| | 26 | L-carnitine + biotin + | 68 | 0.001 | 0.001 | 0.001 |
| | | vitamin E + bisabolol | | | | |
| | 27 | L-carnitine + biotin + | 84 | 0.001 | 0.001 | 0.001 |
| | | vitamin B12 + panthenol | | | | |
| | 28 | L-carnitine + biotin + | 62 | 0.001 | 0.001 | 0.01 |
| | | vitamin E + panthenol | | | | |
| | 29 | L-carnitine + biotin + | 81 | 0.001 | 0.001 | 0.001 |
| | | Vitamin B12 + bisabolol + | | | | |
| | | Panthenol | | | | |
| | 30 | L-carnitine + biotin + | 85 | 0.001 | 0.001 | 0.001 |
| | | Vitamin E + bisabolol + | | | | |
| | | Panthenol | | | | |

The results reported in Table 1 show that the compositions of the invention reduce in a statistically significant manner the onset of the edema when the animals were treated with a combination composition comprising at least three active ingredients mixed together.

The presence of L-carnitine, biotin and a third active ingredient is essential for the activity of the combination composition of the present invention.

In fact, in the groups of mice treated with the combination compositions comprising three active ingredients and in which L-carnitine and/or biotin was not present, the reduction of the edema was not significant respect to the control group (data not shown).

The pharmaceutical or cosmetic composition according to the present invention may be bought with or without medical prescription and is composed of active ingredients which are familiar to operators in the medical field and already in use in clinical practice, and their pharmacotoxicological profiles are known.

Their procurement therefore is very easy, inasmuch as these are products which have been on the market now for a long time and are of a grade suitable for human or animal administration.

L-carnitine and its alkanoyl derivatives are known compounds, the preparation process for which is described in U.S. Pat. No. 4,254,053.

Biotin (The Merck Index, 12th edition (1996), Abstract No. 1272) can be present as a racemate or in optically active form (D- or L-). The D-biotin occurring in nature and/or its derivatives are preferred.

Vitamin B₁₂ and Vitamin E are widely used in medical and cosmetic field.

Bisabolol (CAS-Number: [515-69-5]) is obtained from the chamomile plant.

Panthenol (CAS No.: 16485-10-2) is a non-irritating form of Vitamin B that is usually derived from plants.

Accordingly, cosmetic or topical dermatological compositions in terms of the present invention, depending on their composition, can be used, for example, as a skin protection cream, cleansing milk, sunscreen lotion, nutrient cream, day or night cream etc. It is optionally possible and advantageous to use the compositions according to the invention as a basis for pharmaceutical formulations.

Cosmetic preparations in terms of the present invention can also be present as ointments, creams, lotions baths and solutions powders or gels, which beside an active content of the active ingredient according to the invention and solvents customarily used for this purpose, preferably water, additionally contain organic thickening agents, e.g. gum arabic, xanthan gum, sodium alginate, cellulose derivatives, preferably methyl-cellulose, hydroxymethylcellulose, hydroxyethyl-cellulose, hydroxypropylcellulose, hydroxypropylmethyl-cellulose or inorganic thickening agents, e.g. aluminum silicates such as, for example, bentonites, or a mixture of polyethylene glycol and polyethylene glycol stearate or distearate. The thickening agent is contained in the gel, for example, in an amount between 0.001 and 30% by weight.

In the following are reported a non limiting example of the composition of the present invention.

COMPOSITION 1

| Components | % |
|---|---|
| L-carnitine | 5 |
| Biotin | 1 |
| Vitamin E | 0.5 |
| α-bisabolol | 1 |
| Panthenol | 1 |
| Excipients/diluents | Up to/Till 100 |

COMPOSITION 2

| Component | % |
|---|---|
| L-carnitine | 5 |
| Biotin | 1 |
| Vitamin B12 | 0.1 |
| Excipients/diluents | Up to/Till 100 |

1. Composition, for topical use, comprising as active ingredients L-carnitine and/or one or more alkanoyl L-carnitines, or mixture thereof, or one of their pharmaceutically acceptable salts; biotin; and one ore more of the following active ingredients: vitamin B 12, vitamin E, bisabolol and panthenol.

2. Composition of claim 1, comprising as active ingredients L-carnitine; biotin; vitamin E; bisabolol; and panthenol.

3. Composition of claim 1, comprising as active ingredients L-carnitine; Biotin and vitamin B 12.

4. Composition of claim 1, wherein:
L-carnitine or a derivative thereof is present in an amount of from 0.1 to 20% by weight; or a derivative thereof is present in an amount of from
0.01 to 20% by weight;
vitamin B 12 or a derivative thereof is present in an amount of from 0.001 to 10% by weight;
vitamin E or a derivative thereof is present in an amount of from 0.01 to 10% by weight;
bisabolol or a derivative thereof is present in an amount of from 0.01 to 20% by weight; and
panthenol or a derivative thereof is present in an amount of from 0.01 to 20% by weight.

5-6. (canceled)

7. Method of preventing and/or treating inflammatory and/or degenerative disturbances of the skin with the composition of claim 1 in patients in need thereof comprising
administering to said patients an effective amount of the composition of claim 1 and
treating said patients of said inflammatory and/or degenerative disturbances of the skin.

8. Composition of claim 7, in which the disturbance of the skin is selected from the group consisting of: erythematous, inflammatory, allergic, autoimmune and oxidative disturbance of the skin.

9. Composition of claim 7, in which the disturbance of the skin is selected from the group consisting of: dermatitis or dermatoses.

10. Composition of claim 1, further comprising coenzymes, antioxidants, vitamins, mineral salts and solar filter.

11. Composition of claim 1, in the form of ointments, creams, lotions baths, solutions powders, gels, or in liposome form.

12. Composition of claim 1, in which the pharmaceutically acceptable salt of L-carnitine or alkanoyl L-carnitine is selected from the group consisting of: chloride, bromide, orotate, aspartate, acid aspartate, acid citrate, magnesium citrate, phosphate, acid phosphate, fumarate and acid fumarate, magnesium fumarate, lactate, maleate and acid maleate, oxalate, acid oxalate, pamoate, acid pamoate, sulphate, acid sulphate, glucose phosphate, tartrate and acid tartrate, glycerophosphate, mucate, magnesium tartrate, 2-amino-ethanesulphonate, magnesium 2-amino-ethanesulphonate, methanesulphonate, choline tartrate, trichloroacetate, and trifluoroacetate.
